(19) United States
(12) Patent Application Publication
(10) Pub. No.: US 2005/0230067 A1
(43) Pub. Date: Oct. 20, 2005

(54) Mosquito curtain

(76) Inventors: Elizabeth Jordan, Dunwoody, GA (US); Kurt Jordan, Dunwoody, GA (US)

Correspondence Address: Kurt & Elizabeth Jordan, 5725 Roberts Drive, Dunwoody, GA, 30338, US

(21) Appl. No.: 10/826,625

(22) Filed: Apr. 19, 2004

(51) Int. Cl. A61K 31/55; A01N 43/62; A47H 1/00; E06B 9/56

(52) U.S. Cl. 160/330

(57) ABSTRACT

A removable, washable, and frameless curtain comprising insect netting secured to a valance strip to act as a flying insect barrier when attached to fixed covered outdoor structures such as covered porches, porticos, gazebos and covered pergolas.

### CROSS REFERENCE TO RELATED APPLICATIONS

Not applicable

### FEDERALLY SPONSORED RESEARCH

Not applicable

### SEQUENCE LISTING OR PROGRAM

Not applicable

### BACKGROUND

1. Field of Invention

This invention relates to a physical barrier to mosquitoes and other flying insects to be used primarily for fixed covered outdoor structures subject to flying insect exposure such as covered porches, porticos, gazebos and covered pergolas. (For ease of explanation, this document will use a “porch” as a primary example, however, this invention may apply to any fixed covered outdoor structure.)

2. Description of Prior Art

Mosquito protection is an important health issue. The recurrence of the West Nile Virus and encephalitis highlights the fact that mosquito-borne diseases can originate quickly and are not always easily eradicated.

Mosquito barriers are the most effective prophylactics to mosquito exposure. While many “mosquito killers” such as citronella candles, sprays, zappers, glues, and propane driven trappers are available, nothing is as effective as physically separating mosquitoes from their hosts using a barrier.

Consumers who want to use a barrier to prevent exposure from mosquitoes and other flying insects in a fixed covered outdoor structure will typically build some variation of a wooden frame attached with screening material to create what is commonly known as, a “screened-in porch.” Unfortunately, the screened-in porch is a permanent structure that remains in place year round even during colder seasons when mosquitoes are inactive.

The screening is stretched across the frame and usually stapled. Because it is stretched taut, it is subject to snags and tears requiring periodic screen replacement. Further, due to outside exposure and year round use, the wood frame requires regular painting. In wet areas where mosquitoes are most prevalent, the screen is subject to mildew and requires cleaning and the application of mildew prevention chemicals.

Once a screened in porch is built, the frame becomes a fixed structure that cannot be easily removed. Because of this difficulty, disassembly is not practical should the user decide that an “open” porch is more desirable during cooler months when mosquitoes are inactive.

Because a screened-in porch requires framing and fastening of screen material, it is not easily assembled for an inexperienced consumer. Often, a professional is required to install a screened-in porch. Such costs may make this barrier method prohibitive to a budgeted consumer.

Because a screened-in porch is a year round fixed structure, it is impractical to offer light screen colors such as bright white since the screening material is delicate, fixed in one place, and not thoroughly washable to remove dirt and mildew. Screening materials used for screened-in porches are typically black or gray for this reason. The preferred black and gray colors only camouflage dirt and mildew perhaps giving the consumer a false sense of cleanliness. Consumers sensitive to mold and mildew are more likely to be unaware of mold and mildew with darker colors.

The darkness of the typical screened-in porch changes the look of the porch darkening it from the outside. Also, compared to a bright white alternative, the darker colors block more sunlight into the interior of the dwelling, which may be undesirable. But, because fixed structures have screens not easily removed and washable, they are impractical in light, more easily soiled colors. Under lighted conditions, dark screening materials may be less aesthetically pleasing than light colors.

Restaurants with outside seating under a covered structure often find a screened-in portico impractical since the need for a screened- in portico is typically seasonal. Restaurants are deterred from a permanent structure because the dark colors and framing detract from ambiance and presentation. Restaurants that do not screen-in porticos, subject patrons to flying insects and the occasional harshness of direct sunlight, wind and rain.

A number of U.S. patents act as barriers to outdoor mosquito exposure, but none provide for a frameless curtain that is removable and washable. You will see that the following patents either provide for a stand-alone framed enclosure or a framed enclosure whereby the frame is attached to a structure.

U.S. Pat. No. 6,216,716 (Mosquito Net) provides for mosquito netting attached to an expandable frame. This patent does not accommodate for a curtain that would cover an entire porch area. Further, this patent provides for a built-in frame that would prevent a thorough washing of the netting material that may accumulate dirt and mildew.

U.S. Pat. No. 4,57,852 (Automatic Mosquito Curtain) Is mosquito netting attached to a roller used in windows. This invention also requires a frame and is primarily designed for windows. Such a frame is designed to cover a single plane of exposure and would not accommodate an entire covered structure with multiple planes of exposure. Neither would it be removable and washable.

Other inventions have addressed insect barriers to structures with open exposure. However, the utility of these inventions suffer several disadvantages as they are designed for a garage with only one plane of outside exposure, and they require a frame. In such a case the netting material would not be removable and washable. Also, our invention improves upon these inventions because it can be spanned around corners to cover multiple planes of outside exposure. For example, a gazebo might have six angles and therefore six planes of outside exposure. A single length of a Mosquito Curtain could span an entire length around multiple angles covering multiple planes of exposure.

See:

U.S. Pat. No. 5,899,254 (Garage Screen)
U.S. Pat. No. 5,323,835 (Removable Screen for Garage Door)
U.S. Pat. No. 4,462,183 (Garden Window and Hide-Away Screen
U.S. Pat. No. 4,961,981 (Weighted Netting)

While other solutions have sought to correct individual disadvantages to a screened-in covered structure, none have addressed all of these disadvantages.

As a further note, we were able to find the following patents for curtains with a particular utility function. We believe that our invention also performs a particular utility function, specifically, a curtain acting as a barrier to flying insects. See,
U.S. Pat. No. 5,915,442 (Curtain, More Particularly, A Window Shade)
U.S. Pat. No. 4,288,992 (Curtain For Open Front Freezer or Refrigerator)
U.S. Pat. No. 6,394,171 (Clear Plastic Industrial Curtain)

Remarkably, this invention excels in its simplicity. Despite its simplicity, this invention is unavailable in the current marketplace. This invention will provide a quick, inexpensive, and effective solution to the disadvantages of a permanent screened-in structure.

### OBJECTS AND ADVANTAGES

Accordingly, besides the objects and advantages of the Mosquito Curtain described in our above patent, several objects and advantages of the present invention are:
A) To provide a barrier that is an effective prophylactic to mosquito and other flying insect exposure; and
B) to provide a barrier that is easily removable; and
C) to provide a barrier that effectively seals an enclosure; and
D) to provide a barrier that is more free-flowing than current alternatives and less subject to snags and tears; and
E) to provide a barrier that is aesthetically superior; and
F) to provide a barrier that requires less maintenance; and
G) to provide a barrier that is washable in a washing machine or hand washed in a basin and therefore practical in light colors, including but not limited to white; and
H) to provide a barrier that can tolerate necessary washings to remove dirt and mildew without damage or discoloration; and
I) to provide a barrier that is practical in a light colors such as white to allow for a more aesthetically pleasing look especially under lighted conditions.
J) to provide a barrier that is practical in light colors such as white to more accurately indicate the presence of mold and mildew so that it may be sufficiently cleaned when necessary; and
K) to provide a barrier that is more affordable than current alternatives particularly to lower income budgets; and
L) to provide a barrier that is seamless; and
M) to provide a barrier that is easily installed requiring minimal skills; and
N) to provide a limited barrier to direct sunlight, wind, and rain.

### SUMMARY

In accordance with the present invention, this invention comprises a seamless span of insect netting attached to a sturdy valance, such as, but not limited to, canvas or Velcro, whereby all materials are washable in a washing machine or tub. Because insect- netting material is light and delicate, a sturdy valance is necessary because it must be stretched taught to create an effective seal. This invention would be used primarily as a flying insect barrier for fixed covered outdoor structures such as, but not limited to, covered porches, porticos, gazebos and covered pergolas.

### DRAWINGS

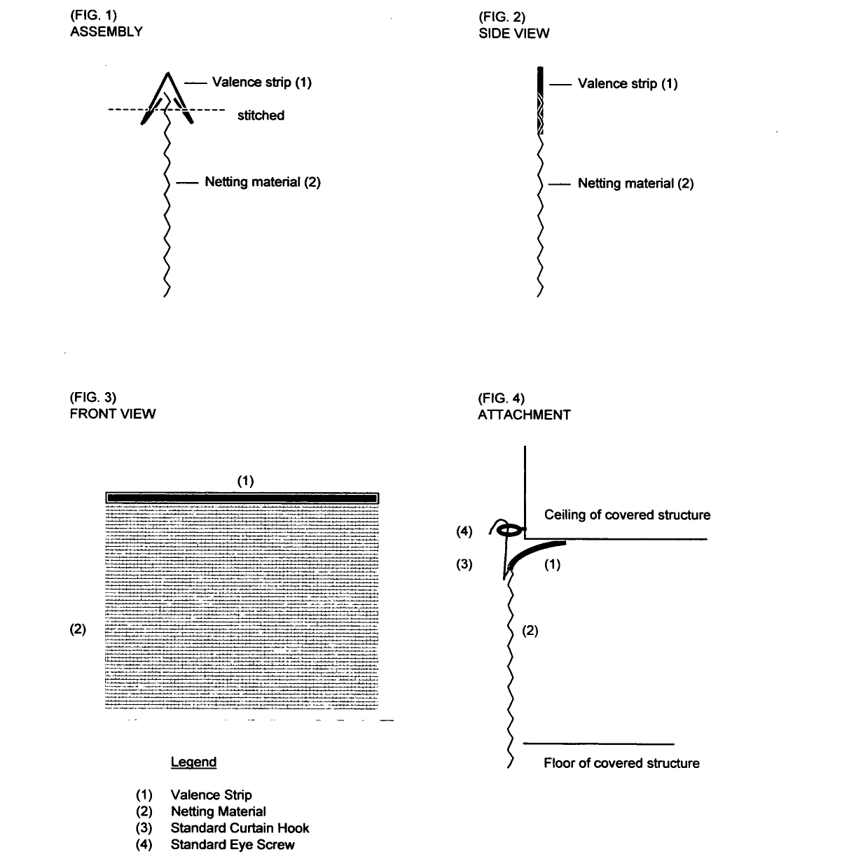

(FIG. 1) shows the configuration of the valance strip relative to the sheet of insect netting.

(FIG. 2) shows a side view of the Mosquito curtain.

(FIG. 3) shows a front view of the mosquito curtain.

(FIG. 4) shows a method of hanging the mosquito curtain by inserting the standard curtain hook through a standard eye-screw and the seal created.

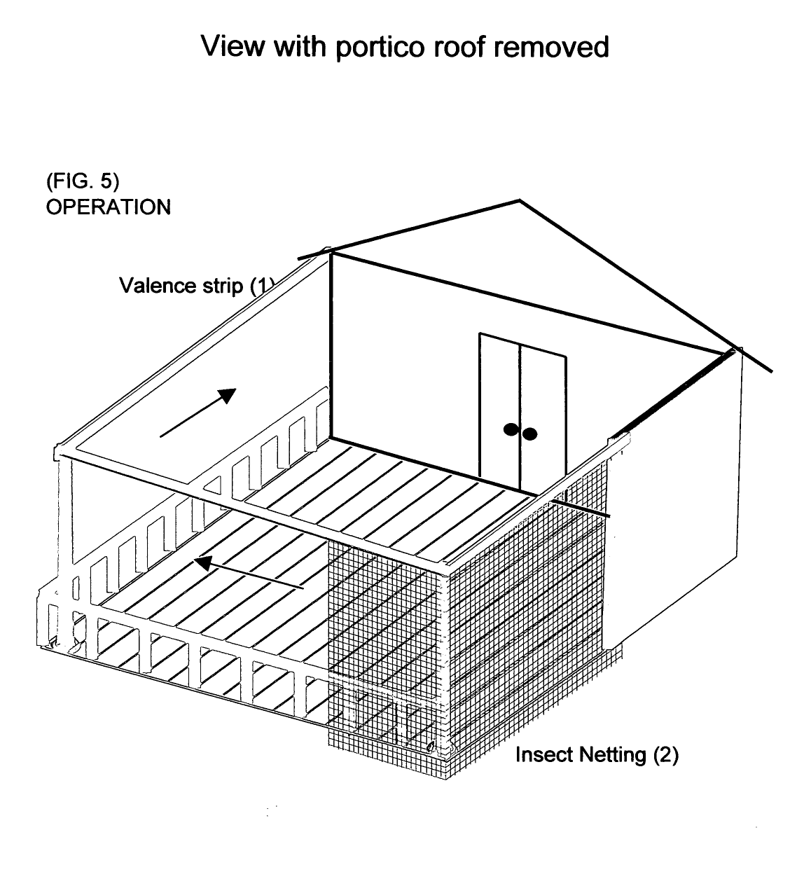

(FIG. 5) shows the operation of the mosquito curtain.

### REFERENCE NUMERALS IN DRAWINGS

(1) Valance strip
(2) Netting material
(3) Standard curtain hook (preferred embodiment)
(4) Standard eye screw (preferred embodiment)
   All items are clearly marked on the drawings.

### DETAILED DESCRIPTION

### Preferred Embodiment

The preferred embodiment is a sheet of insect netting sewn to a sturdy cloth valance strip. Standard curtain hooks are inserted into the seam of the valance strip spaced appropriately and hung onto corresponding eye screws inserted into the outer face adjacent to the ceiling of a porch. The curtain is cut long enough to either drape past the flooring or to rest on the flooring with some excess.

Alternative embodiments (1) and (2) are those that alter the valance strip to accommodate alternative methods of attachment of the mosquito curtain to covered structures. Alternative embodiments (3) and (4) alter the netting material to allow for a passageway through the mosquito curtain.

### Alternative Embodiment (1)

An alternative embodiment would be to sew a Velcro or comparable Velcro-like strip to the valance strip. This embodiment would allow for an alternative form of attachment and would still be removable and washable.

### Alternative Embodiment (2)

An alternative embodiment would be to secure the insect netting directly to a Velcro or Velcro-like strip whereby the Velcro or Velcro-like strip itself is the valance. The Velcro or Velcro-like valance would be secured to the insect netting either by a machine washable adhesive, or by sewing, or both.

### Alternative Embodiment (3)

An alternative embodiment would be to make a vertical cut from the base of the curtain, up to, but not including, the valance to allow passage through the curtain much like a doorway. The cut sides of the doorway could be fastened together in a manner such to secure the fabric edges together when desired to re-seal the desired space from mosquito exposure. Such fastening might be accomplished by attaching a Velcro or Velcro-like strip to the sides of a doorway for re-sealing, or might be accomplished in some other effective manner such as fastening a zipper or cloth ties that would allow for opening and closing of the doorway.

### Alternative Embodiment (4)

An alternative embodiment would be to attach two overlapping sheets of insect netting to a single valance. The sheets would overlap at a point where a passageway through the curtain might be desired. The overlap would extend from the point of attachment to the valance strip to the base of the curtain. The amount of overlap would be sufficient to maintain an effective seal when said doorway is closed.

Advantages

From the description above, a number of advantages of my Mosquito Curtain are evident:
a) It can be easily removed and reinstalled when desired; and,
b) it can be washed while removed from operation such as in a washing machine, or hand washed in a tub; and,
c) it can be offered in light colors such as, but not limited to, bright white and still be practical; and
d) it can span linearly around any shaped fixed outdoor covered structure; and
e) it can be installed easily; and,
f) it is simpler requiring few materials and less expensive than fixed screened-in structures; and,
g) the valance will provide a sturdy material for attachment; and,
h) when stretched around at least one corner, will create integrity of form such that no bottom attachment will be necessary; and,
i) can act as a limited barrier to direct sunlight, wind and rain; and,
j) can accommodate a vertical slit doorway; and
k) is unavailable in the current market place.
   Operation

The Mosquito curtain is hung into place, as described in (FIG. 4) of the diagram. In the preferred embodiment, the valance is attached to the outer edge of the portico ceiling using standard curtain hooks and standard eye screws. In alternative embodiments (1 and 2), the valance is attached to the outer edge of the fixed covered outside structure ceiling using a Velcro or Velcro-like strip.

Unlike framed screening that is taught spanning a relatively small frame, the Mosquito Curtain is pulled taught only at the valance.

The bottom of the mosquito curtain effectively becomes sealed at its base when the top valance is stretched across at least one angle and the insect netting length is cut appropriately. The sides of the curtain can either overlap the portico walls or can be fastened to the wall by sandwiching the netting with a strip of molding, or some other practical method such as a Velcro strip. Once in place, it requires no further operation until the owner wishes to remove it for any reason.

Periodically, the curtain is removed and the curtain hooks are pulled from the valance seam. To wash, the curtain is put into an appropriate sized washing machine or hand washed in a tub. The curtain is drip dried and the valence can be hand ironed if so desired. The curtain may either be stored or re-hung for use.

### CONCLUSION, RAMIFICATIONS, AND SCOPE

Accordingly, the reader will see that the Mosquito Curtain is a cheap alternative to a screened-in covered structure and will create an effective barrier to mosquito exposure. In addition, it is removable and washable. It is much more flexible in its use, maintenance, and aesthetic appeal than current options.

This invention is unavailable in the current market place and an important prophylactic to mosquito-borne health risks particularly for individuals with limited budgets. Furthermore, the Mosquito Curtain has the additional advantage that
It is washable and therefore practical to use light colored mosquito netting such as “white.”
It is aesthetically more pleasing than other alternatives.
It provides an effective prophylactic to flying insects such as mosquitoes.
It is easy to install and easy to remove.
It can span around any shaped fixed covered outdoor structure.
It is more free flowing and snag resistant.
It provides for a limited barrier to direct sunlight wind and rain.
It is currently unavailable in the marketplace.

Although the description above contains several specifications, these should not be construed as limiting the scope of the invention but as merely providing some illustrations of some of the preferred embodiments of this invention. For example, the valance may be made of various sturdy cloth materials or made of a Velcro or Velcro-like strip. Also, colors other than bright white may be used as well as multiple colored netting or netting with design patterns. Also, while throughout this document I have referred to a “porch,” this invention can be used for a variety of covered structures. Also, while I have referred to mosquitoes, this invention can be used as a barrier to most flying insects.

1. A curtain to prevent flying insects from entering a space comprised of screen material secured to a valance strip.

2. A curtain according to claim 1 wherein the curtain is easily removable and washable.

3. A curtain according to claim 1 wherein the said screen is comprised of either mosquito netting or insect netting.

4. A curtain according to claim 1 wherein the valance strip is comprised of any cloth material.

5. A curtain according to claim 1 wherein the valance strip is attached to the screen by means of stitching or by means of an adhesive.

6. A curtain according to claim 1 wherein the valance strip is comprised of Velcro.

7. A curtain according to claim 1 wherein the valance strip is comprised of a Velcro-like material.

8. A curtain according to claim 4 wherein the valance strip is comprised of canvas.

9. A curtain according to claim 4 wherein Velcro is secured to said cloth valance strip for the purpose of attachment to an outdoor structure.

10. A curtain according to claim 4 wherein grommets fastened to said cloth valance strip for the purpose of attachment to an outdoor structure.

11. A curtain according to claim 1 wherein the said space is a fixed covered outdoor structure.

12. A curtain according to claim 11 wherein the said fixed covered outdoor structure is a covered porch, or a gazebo, or a covered pergola, or a portico, or a garage.

13. A curtain according to claim 1 wherein the said screen is cut vertically from its base up to but not including the valance strip to allow for a doorway of passage.

14. A curtain according to claim 13 wherein the two sides of the cut screen are attached with a means to re-seal the doorway of passage.

15. A curtain according to claim 14 wherein the means of re- sealing the doorway of passage uses either a zipper, Velcro, Velcro-like material, clips, hooks, or cloth ties.

16. A curtain according to claim 3 wherein said insect netting or mosquito netting is any color including, but not limited to, bright white.

17. A curtain according to claim 3 wherein said insect netting or mosquito netting has a design pattern.
